(19) United States
(12) Patent Application Publication
(10) Pub. No.: US 2026/0201391 A1
(43) Pub. Date: Jul. 16, 2026

(54) COMPOSITIONS AND METHODS RELATED TO MULTIPARATOPIC APTAMERS

(71) Applicant: North Carolina State University, Raleigh, NC (US)

(72) Inventors: Yi Xiao, Raleigh, NC (US); Obtin Alkhamis, Raleigh, NC (US)

(21) Appl. No.: 19/136,535

(22) PCT Filed: Dec. 8, 2023
(86) PCT No.: PCT/US2023/083134

§ 371 Date: Jun. 6, 2025

Related U.S. Application Data
(60) Provisional application No. 63/386,570, filed on Dec. 8, 2022.

(51) Int. Cl. C12N 15/115; C12N 15/10

(52) U.S. Cl. CPC C12N 15/115; C12N 15/1065; C12N 2310/16

(57) ABSTRACT

The present disclosure provides compositions and methods related to multiparatopic aptamers. In particular, the present disclosure provides nucleic acid aptamers capable of binding multiple distinct epitopes on a target biomolecule, as well as corresponding methods of generating and characterizing the multiparatopic aptamers.

### CROSS REFERENCE TO RELATED APPLICATIONS

This application claims priority to and the benefit of U.S. Provisional Ser. No. 63/386,570 filed Dec. 8, 2022, which is incorporated herein by reference in its entirety and for all purposes.

### FIELD

The present disclosure provides compositions and methods related to multiparatopic aptamers. In particular, the present disclosure provides nucleic acid aptamers capable of binding multiple distinct epitopes on a target biomolecule, as well as corresponding methods of generating and characterizing the multiparatopic aptamers.

### BACKGROUND

There are many chemotherapeutics for the treatment of cancer, which generally target vital processes for cancer cell survival, proliferation, and metastasis such as the cell cycle, growth signaling pathways, or DNA synthesis and repair. Traditional chemotherapeutic drugs are highly potent, but have severe off-target effects due to their inability to differentiate cancer cells from healthy cells. Newer targeted therapies such as tyrosine kinase inhibitors (TKIs) and monoclonal antibodies are now widely applied as either standalone or adjuvant treatment with traditional chemotherapies to improve treatment outcomes through enhanced selectivity. Many targeted drugs inhibit overexpressed or mutated cell signaling proteins that drive tumor development and/or growth. This allows for the selective destruction of diseased cells while largely sparing healthy cells. However, targeted therapies are highly susceptible to acquired resistance. Such resistance is especially common with therapies targeting epidermal growth factor receptor (EGFR), and may arise from overexpression and/or mutations of EGFR, mutational activation of downstream signaling proteins (e.g., KRAS G12C), or exploitation of alternative signaling pathways to sustain cell proliferation and survival (e.g., overexpression of HER2). Some of these resistance mechanisms can be overcome with new, alternate inhibitors (e.g., sotorasib for KRAS G12C) or with established drugs targeting upregulated pathways (e.g., trastuzumab for HER2 overexpression). However, target site mutations that result in the impairment of drug-receptor binding remain a consistently challenging resistance mechanism to durably overcome.

For example, the TKI gefitinib is highly effective in patients with EGFR variant L858R, but the acquisition of a mutation in the target binding site (T790M) renders the drug completely ineffective. Three generations of anti-EGFR TKIs have been developed thus far to address this resistance mechanism, but even the latest generation (i.e., osimertinib) encounters resistance due to new target mutations (C797X). Antibody therapies are similarly rendered ineffective by target site mutations. For instance, 16% of patients treated with the anti-EGFR antibody cetuximab develop the EGFR mutation S492R, which completely impairs cetuximab binding. Administering another antibody that binds a different epitope on EGFR would only temporarily remedy this issue, because resistance could re-emerge through even a single point mutation at its epitope, as has been seen with TKIs for instance.

Several efforts are underway to combat mutation-based resistance to antibody therapies. One approach is to simultaneously target multiple non-overlapping epitopes of the target based on the rationale that individual mutations at one site would not affect the binding affinity—and hence therapeutic efficacy—of drugs recognizing different epitopes on that same target. For example, a novel ‘oligoclonal’ antibody regimen currently in clinical trials employs three antibodies in a mixture that bind different epitopes of the extracellular domain of EGFR, and has encouragingly demonstrated similar inhibitory activity for both wild-type and mutated EGFR. However, oligoclonal antibody therapy faces difficulties in formulation due to differences in the pharmacokinetics of its constituent antibodies and their combined toxicity profiles. To address this problem, one can integrate several affinity elements binding to different epitopes on the same target into a single construct (i.e., a ‘multiparatopic’ binder). This not only synchronizes the pharmacokinetics, but also enhances the binding affinity. For example, biparatopic antibodies that bind two different epitopes on a single target have been developed by linking the N-terminus of a monoclonal antibody heavy chain with another affinity element (e.g., a Fab or scaffold-based binding protein) via a flexible linker. These typically have a 10-100-fold lower k_{off} than standard antibodies while also retaining the pharmacokinetic benefits of whole antibodies. Multiparatopic constructs can also be made by covalent linkage of multiple scaffold-based binding proteins (e.g., affimers or nanobodies), and these typically achieve a 10-fold reduction in K_{D} and k_{off} relative to their individual constituent subunits.

However, protein-based multiparatopic constructs, whether based on antibodies, affimers, or nanobodies, have seen only limited clinical use due to the massively labor-intensive trial-and-error process required to select the best combination of affinity elements and optimal linker domains. They are also costly to produce, prone to batch-to-batch variation, immunogenic, and have short shelf-lives. Thus, there is a great need for new, generally-applicable methodologies for the straightforward generation of high-affinity multiparatopic biomolecules.

### SUMMARY

Embodiments of the present disclosure include a single-stranded nucleic acid molecule comprising an anchor aptamer capable of binding a target biomolecule, and at least one candidate aptamer coupled to the anchor aptamer. In accordance with these embodiments, the at least one candidate aptamer comprises a random nucleic acid sequence.

In some embodiments, the anchor aptamer comprises a pre-determined nucleic acid sequence that binds an epitope on the target biomolecule. In some embodiments, the anchor aptamer comprises a nucleic acid sequence that is from about 10 nucleotides to about 50 nucleotides in length. In some embodiments, the anchor aptamer comprises a nucleic acid sequence of a reference aptamer sequence. In some embodiments, the anchor aptamer comprises a nucleic acid sequence comprising at least one modification as compared to a reference aptamer sequence. In some embodiments, the at least one modification comprises one or more of: (i) a substitution of one or more nucleotides; (ii) a deletion of one or more nucleotides; and/or (iii) an insertion of one or more nucleotides, as compared to a reference aptamer sequence. In some embodiments, the anchor aptamer binds the target biomolecule with a K_{D} that is from about 10 nM to about 10 μM.

In some embodiments, the nucleic acid molecule comprises two candidate aptamers flanking the anchor aptamer. In some embodiments, the at least one candidate aptamer comprises a random nucleic acid sequence that is determined to bind the target biomolecule. In some embodiments, the at least one candidate aptamer binds an epitope on the target biomolecule that is different from the epitope to which the anchor aptamer binds. In some embodiments, the at least one candidate aptamer comprises a nucleic acid sequence that is from about 10 nucleotides to about 50 nucleotides in length.

In some embodiments, the nucleic acid molecule binds the target biomolecule with a higher affinity than a reference anchor aptamer. In some embodiments, the nucleic acid molecule binds the target biomolecule with a K_{D} that is from about 1 fM to about 1 nM. In some embodiments, the nucleic acid molecule is from about 30 nucleotides to about 150 nucleotides in length.

In some embodiments, the nucleic acid molecule is RNA, DNA, or combinations thereof. In some embodiments, the nucleic acid molecule comprises 2′-deoxy-2′-fluoro-ribonucleotides (2′-F RNA), a phosphorothioate modification, a 2′-O Methyl sugar, a LNA (locked nucleic acid), and a threose nucleic acid (TNA), and any combination thereof.

In some embodiments, the nucleic acid molecule further comprises a linker. In some embodiments, the nucleic acid molecule further comprises a linker, and wherein the linker facilitates binding of the nucleic acid molecule to a solid support. In some embodiments, the nucleic acid molecule further comprises a fluorescent tag or an electroactive tag.

In some embodiments, the target biomolecule is a protein or polypeptide. In some embodiments, the target biomolecule is selected from the group consisting of cell surface receptors, endogenous biomarkers, cardiac proteins, oncoproteins, endocrine polypeptide hormones, gastrointestinal peptide hormones, cytokines, viral proteins, bacterial proteins, fungal proteins, and plant proteins.

In some embodiments, the target biomolecule is a protein or polypeptide that is present in a solution and not bound to a solid support.

Embodiments of the present disclosure also include a library comprising a plurality of any of the nucleic acid molecules described herein. In some embodiments, the plurality of nucleic acid molecules comprises at least 10¹⁰ individual nucleic acid molecules, and each sequence of the least one candidate aptamer is distinct.

Embodiments of the present disclosure also include a method of identifying a multiparatopic aptamer comprising exposing a library of candidate multiparatopic aptamers to a target biomolecule, wherein each candidate multiparatopic aptamer in the library comprises an anchor aptamer capable of binding the target biomolecule and at least one candidate aptamer coupled to the anchor aptamer, wherein the at least one candidate aptamer comprises a random nucleic acid sequence; and assessing binding of the candidate multiparatopic aptamer to the target biomolecule.

In some embodiments, the library of candidate multiparatopic aptamers is exposed to the target biomolecule in multiple rounds of selection. In some embodiments, the multiple rounds of selection comprise exposing the library of candidate multiparatopic aptamers to a variant of the target biomolecule in each round.

In some embodiments, the target biomolecule is a protein or polypeptide. In some embodiments, the protein or polypeptide is expressed by a cell. In some embodiments, the protein or polypeptide is conjugated to a solid support.

In some embodiments, assessing binding of the candidate multiparatopic aptamer to the target biomolecule comprises determining whether the K_{D} of the candidate multiparatopic aptamer is below a threshold. In some embodiments, the threshold K_{D} is at least 10-fold lower than the K_{D} of the anchor aptamer.

In some embodiments, assessing binding of the candidate multiparatopic aptamer to the target biomolecule comprises performing surface plasmon resonance, isothermal titration calorimetry, microscale thermophoresis, biolayer interferometry, strand-displacement fluorescence assay, and intrinsic tryptophan fluorescence quenching, and any combination thereof.

### BRIEF DESCRIPTION OF THE DRAWINGS

FIG. 1: Representative schematic diagrams illustrating the structure and affinity of thrombin aptamers Tasset-29 and Bock-15, and biparatopic aptamer TBV-08.

FIG. 2: Advantages of multiparatopic aptamers of the present disclosure: unlike monoparatopic binders (left), multiparatopic binders have ultra-high affinities and can tolerate single-site mutations due to binding of multiple epitopes (right).

FIGS. 3A-3C: Isolating a multiparatopic binding aptamer for the extracellular domain of cell-surface hEGFR using a structured 2′F-RNA library. Three-dimensional structure of monomeric hEGFR as determined via X-ray crystallography (PDB: 3QWQ), embedded in a phospholipid bilayer (gray) via its transmembrane domain (PDB: 2KS1) (FIG. 3A). Side and top views are shown at left and right, respectively. Schematic of hEGFR extracellular domain organization (FIG. 3B). In A and B, domains I, II, III, and IV are respectively colored gold, magenta, green, and blue. The reported hEGFR aptamer (red anchor) binds to domain III, and is flanked by binding domains at both termini (black lines). Sequence and structure of the proposed 2′-F RNA Anchor-SELEX library (FIG. 3C). Region 1 (R1) is the anchor aptamer, which will be doped at a ratio of 2:1 original nucleotide vs. alternative nucleobases. Regions 2 and 3 (R2 and R3) are 20-nt random regions at the 5′and 3′ends, respectively, that serve as additional binding domains for other epitopes on hEGFR.

FIGS. 4A-4B: Strategy for Anchor-SELEX. Anchor-SELEX process, including target cell lines and hEGFR variants used in each selection round (FIG. 4A). Structure of hEGFR, with the various mutants highlighted in red (FIG. 4B).

FIG. 5: Binding affinity of the multivalent aptamer versus the monoclonal antibody cetuximab and anchor aptamer, ranked from highest to lowest affinity. A single-site hEGFR mutation may impair triparatopic aptamer binding, but it should retain affinity better than or equal to that of cetuximab.

FIG. 6: Monoparatopic and multiparatopic constructs derived from the triparatopic aptamer for affinity testing.

### DETAILED DESCRIPTION

Embodiments of the present disclosure have a systematic approach for selecting aptamers that simultaneously bind to multiple, independent epitopes on a target protein, which has been termed “multiparatopic” aptamers. Such aptamers will be of great value for both clinical chemistry and the development of new, highly targeted therapies. For the former, the greatly enhanced affinity of such aptamers will translate to improved limits of detection for pathogens and other low-concentration analytes. Additionally, multiparatopic aptamers could also be important for the development of novel therapies, such as novel cancer therapies.

Aptamers are a promising candidate in this regard. Generally, aptamers are single-stranded nucleic acid-based affinity reagents isolated from randomized libraries in vitro through various processes, including but not limited to, the use of systematic evolution of ligands by exponential enrichment (i.e., SELEX) to bind specific targets with high affinity. Aptamers have several desirable properties as therapeutics, including high affinity and specificity, low toxicity, low immunogenicity, efficient tissue penetration, and the ease at which they can be chemically modified to achieve improved half-life and high chemical stability in vivo.

In particular, two factors make aptamers ideal for developing multiparatopic agents. First, aptamers are modular and highly amenable to sequence engineering, allowing the coupling of multiple aptamer domains into a single construct that binds multiple epitopes on a single target. Biparatopic aptamers, in which two different aptamers are joined with a polynucleotide linker, have shown 10-50 fold improvement in target affinity. The second advantage is that SELEX enables the screening of vast, highly diverse libraries of oligonucleotides (~10¹⁵ unique sequences), and selection pressure can be applied to isolate the most optimal multiparatopic aptamers with desired binding properties. This is in sharp contrast to the engineering of biparatopic antibodies or aptamers, which entails trial-and-error testing of a handful of constructs with a virtually infinite number of linker and binding domain combinations.

As described further herein, by optimizing only the linker domain of a biparatopic thrombin-binding aptamer using SELEX, target affinity was improved by at least 200-fold. Given these advantages, experiments can be conducted to develop a new selection technique termed “Anchor-SELEX” to generate multiparatopic aptamers that bind to specific targets with ultra-high affinity and retain tight binding even if single target-site mutations occur. This method utilizes a structured library, in which a known target-binding aptamer (e.g., a reference aptamer) that acts as an “anchor” is flanked by random sequences that serve as additional binding domains. This library is subjected to several rounds of selection to isolate high-affinity aptamers with multiparatopic binding capability for a target of interest.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “aptamer” generally refers to either an oligonucleotide of a single defined sequence or a mixture of said oligonucleotides, wherein the mixture retains the properties of binding specifically to a target molecule. Thus, as used herein “aptamer” denotes both singular and plural sequences of oligonucleotides. The term “aptamer” generally refers to a single-stranded that is capable of binding to a protein or other molecule, and thereby modulating function.

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. “Oligonucleotide” or “oligomer” can also be used to describe artificially synthesized polymers that are similar to RNA and DNA, including, but not limited to, oligos of peptide nucleic acids (PNA).

The terms “binding activity” and “binding affinity” generally refer to the tendency of a ligand molecule to bind or not to bind to a target. The energetics of these interactions are significant in “binding activity” and “binding affinity” because they can include definitions of the concentrations of interacting partners, the rates at which these partners are capable of associating, and the relative concentrations of bound and free molecules in a solution.

“Complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, to place the elements in the proper conformation for complementarity, to co-localize complementary elements, to lower interaction energy for complementation, etc.

As used herein, the terms “nucleotide sequence identity” or “nucleic acid sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

### 2. Multiparatopic Aptamers

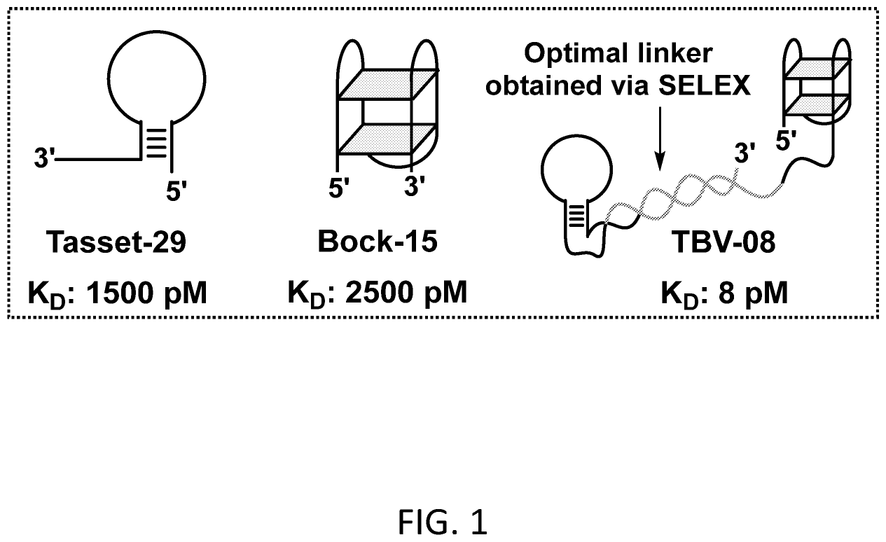

In accordance with the various embodiments of the present disclosure, described herein are methods and compositions pertaining to the identification of multiparatopic nucleic acid molecules capable of binding multiple epitopes on a given target biomolecule. As proof of concept, previous experiments utilizing SELEX identified an optimal linker sequence of a biparatopic DNA aptamer from a library of oligonucleotides containing two previously reported monoparatopic thrombin-binding aptamers (Tasset-29 and Bock-15) connected by a 35-nt random region. After five rounds of selection, the isolated aptamer (TBV-08) had a K_{D} of 8 pM for thrombin, which is respectively 200- and 300-fold lower than the original Tasset-29 and Bock-15 aptamers (FIG. 1). In contrast, 10²¹ linker domain sequences would need to be designed and tested to identify such an aptamer through brute force engineering and characterization. Notably, the affinity of TBV-08 is several orders of magnitude improved relative to other biparatopic aptamers that were engineered from Tasset-29 and Bock-15. In addition, TBV-08 inhibited thrombin with 80-fold greater potency in a fibrinogen cleavage activity assay relative to Bock-15, presumably because its biparatopic binding increases affinity. These results support the feasibility of using Anchor-SELEX to isolate, rather than engineer, high-affinity multiparatopic aptamers for protein targets.

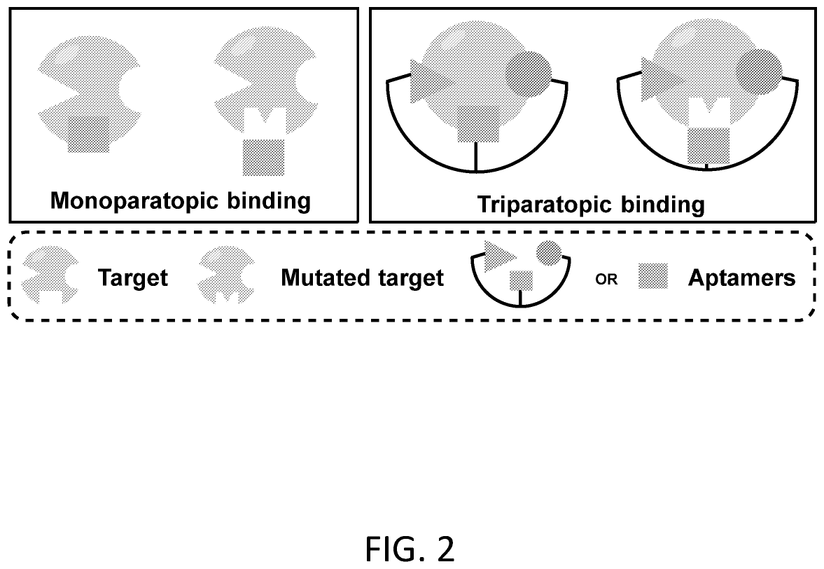

In accordance with the above, embodiments of the present disclosure involve the development of a generalizable SELEX approach for the isolation of high affinity multiparatopic aptamers. For example, multiparatopic aptamers can be identified that serve as novel targeted cancer therapeutics that circumvent drug resistance by target mutations. In one embodiment, a therapeutic can be based on a multiparatopic 2′-fluoro (2′-F) RNA aptamer that binds to the extracellular domain of human EGFR (hEGFR) on cells. Aberrantly activated in many tumors, hEGFR is a well-established target for colorectal, lung, skin, and head and neck cancer. A previously reported monoparatopic 2′-F-modified RNA aptamer can be utilized (e.g., a reference anchor aptamer sequence) that binds cell-surface hEGFR with a K_{D} of 50 nM as a scaffold to construct a library containing randomized domains at both termini as putative binding sites that recognize additional epitopes on hEGFR. The core aptamer scaffold thus serves as an “anchor” for the two additional aptamer domains, which wrap around the periphery of the target to further stabilize and enhance binding. By enabling recognition of multiple target domains, this multiparatopic binding confers robust tolerance to individual mutations relative to conventional aptamers (FIG. 2). Quantitatively, the isolated multiparatopic aptamer can have a K_{D} in the range of ~50 pM, which is about 1,000-fold lower than the original monoparatopic aptamer. Even if a single target site mutation occurs, the remaining two aptameric domains still bind the target with similar affinity as the FDA-approved drug cetuximab (K_{D}<1 nM).

Multiparatopic aptamer sequences can also be identified and their binding capability, stoichiometry, affinity, and kinetics in binding to free extracellular hEGFR can be characterized. Similar analysis can be performed on various cancer cell lines harboring hEGFR or its mutant isoforms. Experiments can evaluate the potential therapeutic efficacy of the best aptamer in vitro using various cancer cell lines, perform mechanistic studies of how aptamer-EGFR binding inhibits cell proliferation and survival, and compare aptamer performance to standard EGFR-targeting antibody therapies. These data can be the basis for the generation of multiparatopic aptamers for other protein biomarkers, preparation of these aptamers for in vivo use by tuning their pharmacokinetics (e.g., conjugation to polyethylene glycol, gold nanoparticles, or Fc protein to increase biological half-life), and evaluation of their safety and efficacy as therapeutic agents in vivo. Importantly, such high affinity receptors can also be utilized as novel diagnostic reagents, with the capability to detect ultra-low levels of medically important analytes (e.g., pathogens or endogenous biomarkers).

As described herein, embodiments of the present disclosure include the use of the cell-SELEX technique with a structured 2′-F RNA oligonucleotide library to isolate a high-affinity multiparatopic aptamer for cell-surface hEGFR. 2′-F RNA is highly resistant to nuclease digestion, and such aptamers have been successfully applied to in vivo applications. To ensure that the isolated aptamer can tolerate individual target site mutations, Anchor-SELEX can be used to isolate aptamers that bind hEGFR simultaneously at multiple sites and a “toggle” selection strategy against different cell lines expressing wild-type hEGFR or its mutants. However, as would be recognized by one of ordinary skill in the art, other types of SELEX methods can be used with the various embodiments of the present disclosure, including target-immobilized SELEX, library-immobilized SELEX, nuclease-assisted SELEX and filter SELEX.

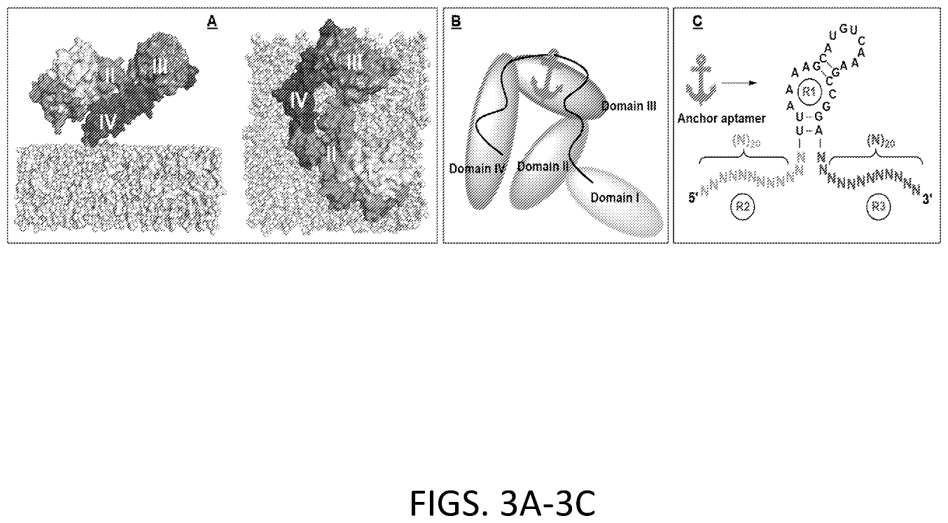

For example, the extracellular region of hEGFR contains four different domains. In the inactive “tethered” conformation depicted in FIG. 3A, domains I and III serve as binding sites for human epidermal growth factor (hEGF) and other ligands. Domains II and IV form an intermolecular interaction (or tether) in the unliganded monomer (FIG. 3A). In the ligand bound dimer, the receptor adopts a very different conformation and domain II forms the primary dimerization interface. The library design for Anchor-SELEX incorporates a well-characterized aptamer that binds hEGFR on cancer cells via interaction with target domain III with K_{D}~50 nM as a core “anchor,” flanked by two randomized regions that form additional interactions with hEGFR (FIG. 3B). Previous work indicates that the functional binding region of this aptamer is a stretch of 22 nucleotides (nt) located in the middle of the sequence. Therefore, to minimize the length of the final multiparatopic aptamer, this 22-nt motif is used as the anchor. Each RNA library molecule comprises three regions, flanked by PCR primer sites at both ends (FIG. 3C). Region 1 (black) consists of the 22-nt anchor aptamer sequence, where each nucleotide position is ‘doped’ (i.e., partially randomized) such that there is a 67% likelihood of retaining the original nucleobase. This is done to ensure that the final aptamer can recognize hEGFR with mutations at domain III. Regions 2 (pink) and 3 (blue) are fully randomized 20-nt sequences that act as additional linker and binding sites to other epitopes on hEGFR. The length of the random region should be sufficient to enable the formation of high-affinity binding domains with optimal spacing, as most high-affinity protein-binding aptamers on average have only 3-8 nt interacting with the target. Since the anchor targets domain III, the two peripheral sites could enable binding to adjacent areas on domain III or domains II and IV.

In accordance with the above description, embodiments of the present disclosure include a single-stranded nucleic acid molecule comprising an anchor aptamer capable of binding a target biomolecule, and at least one candidate aptamer coupled to the anchor aptamer. In some embodiments, the at least one candidate aptamer comprises a random nucleic acid sequence.

In some embodiments, the anchor aptamer comprises a pre-determined nucleic acid sequence that binds an epitope on the target biomolecule (e.g., a reference aptamer sequence). In some embodiments, the anchor aptamer comprises a nucleic acid sequence that is from about 10 nucleotides to about 50 nucleotides in length. In some embodiments, the anchor aptamer comprises a nucleic acid sequence that is from about 10 nucleotides to about 45 nucleotides in length. In some embodiments, the anchor aptamer comprises a nucleic acid sequence that is from about 10 nucleotides to about 40 nucleotides in length. In some embodiments, the anchor aptamer comprises a nucleic acid sequence that is from about 10 nucleotides to about 35 nucleotides in length. In some embodiments, the anchor aptamer comprises a nucleic acid sequence that is from about 10 nucleotides to about 30 nucleotides in length. In some embodiments, the anchor aptamer comprises a nucleic acid sequence that is from about 10 nucleotides to about 25 nucleotides in length. In some embodiments, the anchor aptamer comprises a nucleic acid sequence that is from about 10 nucleotides to about 20 nucleotides in length. In some embodiments, the anchor aptamer comprises a nucleic acid sequence that is from about 10 nucleotides to about 15 nucleotides in length. In some embodiments, the anchor aptamer comprises a nucleic acid sequence that is from about 15 nucleotides to about 50 nucleotides in length. In some embodiments, the anchor aptamer comprises a nucleic acid sequence that is from about 20 nucleotides to about 50 nucleotides in length. In some embodiments, the anchor aptamer comprises a nucleic acid sequence that is from about 25 nucleotides to about 50 nucleotides in length. In some embodiments, the anchor aptamer comprises a nucleic acid sequence that is from about 30 nucleotides to about 50 nucleotides in length. In some embodiments, the anchor aptamer comprises a nucleic acid sequence that is from about 35 nucleotides to about 50 nucleotides in length. In some embodiments, the anchor aptamer comprises a nucleic acid sequence that is from about 40 nucleotides to about 50 nucleotides in length. In some embodiments, the anchor aptamer comprises a nucleic acid sequence that is from about 45 nucleotides to about 50 nucleotides in length. In some embodiments, the anchor aptamer comprises a nucleic acid sequence that is from about 20 nucleotides to about 40 nucleotides in length. In some embodiments, the anchor aptamer comprises a nucleic acid sequence that is from about 15 nucleotides to about 30 nucleotides in length.

In some embodiments, the anchor aptamer comprises a nucleic acid sequence of a reference aptamer sequence. In some embodiments, the anchor aptamer comprises a nucleic acid sequence that is at least 90% identical to a reference aptamer sequence. In some embodiments, the anchor aptamer comprises a nucleic acid sequence that is at least 95% identical to a reference aptamer sequence. In some embodiments, the anchor aptamer comprises a nucleic acid sequence that is at least 98% identical to a reference aptamer sequence. In some embodiments, the anchor aptamer comprises a nucleic acid sequence that is 100% identical to a reference aptamer sequence. In some embodiments, the anchor aptamer comprises a nucleic acid sequence comprising at least one modification as compared to a reference aptamer sequence. In some embodiments, the at least one modification comprises one or more of: (i) a substitution of one or more nucleotides; (ii) a deletion of one or more nucleotides; and/or (iii) an insertion of one or more nucleotides (and any combination thereof) as compared to a reference aptamer sequence. In some embodiments, the modification comprises a substitution of one or more nucleotides as compared to a reference aptamer sequence. In some embodiments, the modification comprises a deletion of one or more nucleotides as compared to a reference aptamer sequence. In some embodiments, the modification comprises an insertion of one or more nucleotides as compared to a reference aptamer sequence.

In some embodiments, the anchor aptamer binds the target biomolecule with a K_{D} that is from about 10 nM to about 10 μM. In some embodiments, the anchor aptamer binds the target biomolecule with a K_{D} that is from about 10 nM to about 5 μM. In some embodiments, the anchor aptamer binds the target biomolecule with a K_{D} that is from about 10 nM to about 1 μM. In some embodiments, the anchor aptamer binds the target biomolecule with a K_{D} that is from about 10 nM to about 500 nM. In some embodiments, the anchor aptamer binds the target biomolecule with a K_{D} that is from about 10 nM to about 250 nM. In some embodiments, the anchor aptamer binds the target biomolecule with a K_{D} that is from about 10 nM to about 100 nM. In some embodiments, the anchor aptamer binds the target biomolecule with a K_{D} that is from about 10 nM to about 50 nM. In some embodiments, the anchor aptamer binds the target biomolecule with a K_{D} that is from about 20 nM to about 10 μM. In some embodiments, the anchor aptamer binds the target biomolecule with a K_{D} that is from about 100 nM to about 10 μM. In some embodiments, the anchor aptamer binds the target biomolecule with a K_{D} that is from about 250 nM to about 10 μM. In some embodiments, the anchor aptamer binds the target biomolecule with a K_{D} that is from about 500 nM to about 10 μM. In some embodiments, the anchor aptamer binds the target biomolecule with a K_{D} that is from about 1 μM to about 10 μM. In some embodiments, the anchor aptamer binds the target biomolecule with a K_{D} that is from about 5 μM to about 10 μM. In some embodiments, the anchor aptamer binds the target biomolecule with a K_{D} that is from about 500 nM to about 5 μM. In some embodiments, the anchor aptamer binds the target biomolecule with a K_{D} that is from about 100 nM to about 1 μM. In some embodiments, the anchor aptamer binds the target biomolecule with a K_{D} that is from about 250 nM to about 2.5 μM. In some embodiments, the anchor aptamer binds the target biomolecule with a K_{D} that is from about 750 nM to about 5 μM.

In some embodiments, the nucleic acid molecule comprises two candidate aptamers flanking the anchor aptamer. In some embodiments, the at least one candidate aptamer comprises a random nucleic acid sequence that is determined to bind the target biomolecule. In some embodiments, the at least one candidate aptamer binds an epitope on the target biomolecule that is different from the epitope to which the anchor aptamer binds.

In some embodiments, the at least one candidate aptamer comprises a nucleic acid sequence that is from about 10 nucleotides to about 50 nucleotides in length. In some embodiments, the at least one candidate aptamer comprises a nucleic acid sequence that is from about 10 nucleotides to about 45 nucleotides in length. In some embodiments, the at least one candidate aptamer comprises a nucleic acid sequence that is from about 10 nucleotides to about 40 nucleotides in length. In some embodiments, the at least one candidate aptamer comprises a nucleic acid sequence that is from about 10 nucleotides to about 35 nucleotides in length. In some embodiments, the at least one candidate aptamer comprises a nucleic acid sequence that is from about 10 nucleotides to about 30 nucleotides in length. In some embodiments, the at least one candidate aptamer comprises a nucleic acid sequence that is from about 10 nucleotides to about 25 nucleotides in length. In some embodiments, the at least one candidate aptamer comprises a nucleic acid sequence that is from about 10 nucleotides to about 20 nucleotides in length. In some embodiments, the at least one candidate aptamer comprises a nucleic acid sequence that is from about 10 nucleotides to about 15 nucleotides in length. In some embodiments, the at least one candidate aptamer comprises a nucleic acid sequence that is from about 15 nucleotides to about 50 nucleotides in length. In some embodiments, the at least one candidate aptamer comprises a nucleic acid sequence that is from about 20 nucleotides to about 50 nucleotides in length. In some embodiments, the at least one candidate aptamer comprises a nucleic acid sequence that is from about 25 nucleotides to about 50 nucleotides in length. In some embodiments, the at least one candidate aptamer comprises a nucleic acid sequence that is from about 30 nucleotides to about 50 nucleotides in length. In some embodiments, the at least one candidate aptamer comprises a nucleic acid sequence that is from about 35 nucleotides to about 50 nucleotides in length. In some embodiments, the at least one candidate aptamer comprises a nucleic acid sequence that is from about 40 nucleotides to about 50 nucleotides in length. In some embodiments, the at least one candidate aptamer comprises a nucleic acid sequence that is from about 45 nucleotides to about 50 nucleotides in length. In some embodiments, the at least one candidate aptamer comprises a nucleic acid sequence that is from about 20 nucleotides to about 40 nucleotides in length. In some embodiments, the at least one candidate aptamer comprises a nucleic acid sequence that is from about 15 nucleotides to about 30 nucleotides in length.

In some embodiments, the nucleic acid molecule binds the target biomolecule with a higher affinity than a reference anchor aptamer. In some embodiments, the nucleic acid molecule binds the target biomolecule with a K_{D} that is from about 1 fM to about 1 nM. In some embodiments, the nucleic acid molecule binds the target biomolecule with a K_{D} that is from about 500 fM to about 1 nM. In some embodiments, the nucleic acid molecule binds the target biomolecule with a K_{D} that is from about 1 pM to about 1 nM. In some embodiments, the nucleic acid molecule binds the target biomolecule with a K_{D} that is from about 500 pM to about 1 nM. In some embodiments, the nucleic acid molecule binds the target biomolecule with a K_{D} that is from about 1 fM to about 500 pM. In some embodiments, the nucleic acid molecule binds the target biomolecule with a K_{D} that is from about 1 fM to about 1 pM. In some embodiments, the nucleic acid molecule binds the target biomolecule with a K_{D} that is from about 1 fM to about 500 fM. In some embodiments, the nucleic acid molecule binds the target biomolecule with a K_{D} that is from about 500 fM to about 500 pM.

In some embodiments, the nucleic acid molecule is from about 30 nucleotides to about 150 nucleotides in length. In some embodiments, the nucleic acid molecule is from about 30 nucleotides to about 125 nucleotides in length. In some embodiments, the nucleic acid molecule is from about 30 nucleotides to about 100 nucleotides in length. In some embodiments, the nucleic acid molecule is from about 30 nucleotides to about 75 nucleotides in length. In some embodiments, the nucleic acid molecule is from about 30 nucleotides to about 50 nucleotides in length. In some embodiments, the nucleic acid molecule is from about 50 nucleotides to about 150 nucleotides in length. In some embodiments, the nucleic acid molecule is from about 75 nucleotides to about 150 nucleotides in length. In some embodiments, the nucleic acid molecule is from about 100 nucleotides to about 150 nucleotides in length. In some embodiments, the nucleic acid molecule is from about 125 nucleotides to about 150 nucleotides in length. In some embodiments, the nucleic acid molecule is from about 75 nucleotides to about 125 nucleotides in length. In some embodiments, the nucleic acid molecule is from about 50 nucleotides to about 100 nucleotides in length.

In some embodiments, the nucleic acid molecule is RNA, DNA, or combinations thereof. In some embodiments, the nucleic acid molecule comprises 2′-deoxy-2′-fluoro-ribonucleotides (2′-F RNA), a phosphorothioate modification, a 2′-O Methyl sugar, a LNA (locked nucleic acid), and a threose nucleic acid (TNA), and any combination thereof. In some embodiments, the nucleic acid molecule comprises 2′-deoxy-2′-fluoro-ribonucleotides (2′-F RNA). In some embodiments, the nucleic acid molecule comprises a phosphorothioate modification. In some embodiments, the nucleic acid molecule comprises a 2′-O Methyl sugar. In some embodiments, the nucleic acid molecule comprises an LNA (locked nucleic acid). In some embodiments, the nucleic acid molecule comprises a threose nucleic acid (TNA).

In some embodiments, the nucleic acid molecule further comprises a linker. In some embodiments, the nucleic acid molecule further comprises a linker, and the linker facilitates binding of the nucleic acid molecule to a solid support. The solid support can be any solid material to which the multiparatopic aptamers of the present disclosure can be grafted and can optionally include pores (e.g., those useful as a stationary phase/packing material for chromatography). In one example, the solid support includes inorganic (e.g., silica) material. In another example, the solid support includes organic (e.g., polymeric) material (e.g., synthetic resins or agarose). In yet another example, the solid support includes a hybrid inorganic-organic material. In one embodiment, the solid support includes metal oxides, metalloid oxides, or magnetic iron oxides (e.g., magnetic particles). Exemplary substrates include silica-based (e.g., silicon oxide, SiO₂), titania-based (e.g., titanium oxide, TiO₂), germanium-based (e.g., germanium oxide), zirconia-based (e.g., zirconium oxide, ZrO₂), alumina-based (e.g., aluminum oxide, Al₂O₃) materials or mixtures thereof. Other substrates include cross-linked and non-crosslinked polymers, carbonized materials and metals. Substrates can also incorporate polymeric networks, sol-gel networks or hybrid forms thereof. In one embodiment, the substrate is a silica-based substrate. Exemplary silica-based substrates include silica gel, glass, sol-gels, polymer/sol-gel hybrids, core-shell structures and silica monolithic materials.

In some embodiments, the nucleic acid molecule further comprises a fluorescent tag or an electroactive tag. The fluorescent tag or an electroactive tag can be any tag known in the art. In some embodiments, the fluorescent tag can include, but is not limited to, green fluorescent protein (GFP), GFP derivatives, such as, Red (RFP), Blue (BFP), Cyan (CFP), Yellow (YFP), Coumarin dyes, Cascade Blue, CyDyes, Fluorescein, Bodipy dyes, Rhodamine (and its derivatives), Allophycocyanin, Alexa Fluor dyes, Texas Red, Lucifer Yellow, TruRed, or Peridinin chlorophyll protein. In other embodiments where fluorescent detection is not used, other types of tags or reporter moieties may be used, such as tags that facilitate electronic detection or radiographic imaging. In some embodiments, the tag comprises a redox tag (e.g., methylene blue), which can be used for electrochemical-based sensors.

In some embodiments, the target biomolecule is a protein or polypeptide. In some embodiments, the target biomolecule is selected from the group consisting of cell surface receptors, endogenous biomarkers, cardiac proteins, oncoproteins, endocrine polypeptide hormones, gastrointestinal peptide hormones, cytokines, viral proteins, bacterial proteins, fungal proteins, and plant proteins. As would be recognized by one of skill in the art based on the present disclosure, the target molecule can include any peptide, polypeptide, or protein of interest, as the multiparatopic aptamers described herein can be selected to bind any epitope.

In accordance with these embodiments, the present disclosure also includes a library comprising a plurality of any of the nucleic acid molecules described herein. In some embodiments, the plurality of nucleic acid molecules comprises at least 1010 individual nucleic acid molecules, and each sequence of the least one candidate aptamer is distinct. In some embodiments, the multiparatopic aptamer library of the present disclosure includes a plurality of the aptamers described above. In some embodiments, the scale of the nucleic acid aptamer library of the present invention is 10¹⁰ or more, 10¹¹ or more, 10¹² or more, 10¹³ or more, 10¹⁴ or more, or 10¹⁵ or more.

### 3. Methods of Identifying Multiparatopic Aptamers

Embodiments of the present disclosure also include methods for isolating a multiparatopic aptamer that binds different epitopes of monomeric cell-surface hEGFR in its native conformation. As described above, experiments can be conducted using the well-established cell-SELEX technique with the designed library, with hEGFR-expressing cancer cell lines as targets. However, as would be recognized by one of ordinary skill in the art, other types of SELEX methods can be used with the various embodiments of the present disclosure, including target-immobilized SELEX, library-immobilized SELEX, nuclease-assisted SELEX, and filter SELEX.

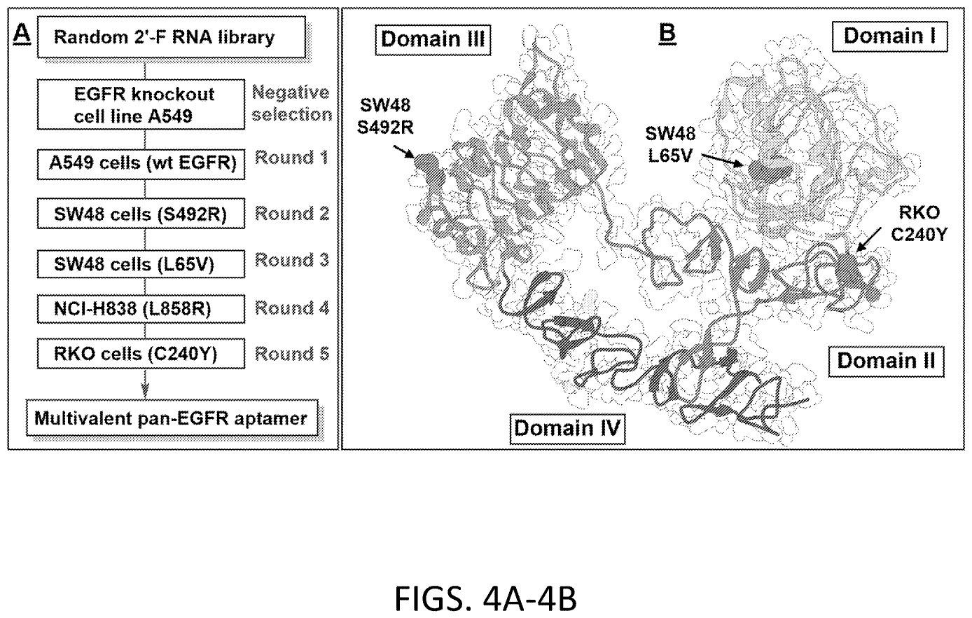

In one embodiment, a negative selection is first performed with A549 EGFR knockout cells to remove non-specific binders in the library. Then, the collected pool is used to perform five rounds of “toggle” selection with multiple cell lines (FIG. 4A) expressing wild-type or mutant hEGFR (FIG. 4B) to ensure that the isolated aptamer can cross-react to all hEGFR variants. In the first round, the library is challenged with A549 cells which overexpress wild-type hEGFR. Since each library molecule contains an anchor that binds domain III of hEGFR, the library can primarily evolve towards enrichment of aptamers that bind this target rather than other cell-surface receptors. In subsequent rounds, four different cell lines expressing hEGFR point mutants are used as targets: SW48 cells with S492R in domain III (Round 2); SW48 cells with L65V in domain I (Round 3); NCI-H1975 with intracellular L858R, which promotes ligand-independent receptor dimerization (Round 4); and RKO cells with C240Y in domain II (Round 5). To enrich the highest-affinity aptamers, each subsequent round employs cell lines expressing decreased levels of hEGFR (i.e., A549 and RKO respectively have the highest and lowest EGFR expression). This process ultimately enriches aptamers that can bind both wild-type and mutant hEGFR. After five rounds, the affinity and cross-reactivity of the aptamer pool to all target cell lines can be assessed, and the pool exhibits high affinity for all cell lines.

Anchor-SELEX is performed using a modified version of a previously reported protocol. For example, the selection targets can include five different human cancer cell lines that overexpress wild-type or mutant hEGFR. The targets are alternated every round of SELEX to isolate multiparatopic aptamers that cross-react to both wild-type and mutated hEGFR. To begin, a DNA library including a 5′ T7 promoter site is in vitro transcribed to generate the 2′-F RNA library (FIG. 3C) using the DuraScribe T7 Transcription Kit. Then, 10 nmol of the 2′-F RNA library (~10¹⁵ unique sequences) is heated to 85° C., cooled on ice to allow the library strands to correctly fold, and then resuspended in 800 μL binding buffer (Dulbecco's phosphate-buffered saline (DPBS; pH 7.4), 5 mM MgCl₂, 25 mM glucose, 0.1 mg/ml yeast tRNA, and 1 mg/ml BSA). Excess tRNA is used to prevent non-specific adsorption of library strands by the cells. Each cell line is cultured in a 10-cm dish to 90% confluence (~10⁷ cells). Prior to adding the library, the cell culture medium is removed, and the cells are washed with washing buffer (binding buffer without tRNA and BSA). The library is then incubated with the cells for 1 hour at 37° C. in the absence of hEGF. After incubation, the supernatant containing unbound library strands is removed and discarded; this also includes oligos that misfold and disrupt the anchor domain. The cells are washed, and then collected by scraping the dish and rinsing with 2 mL of DPBS. This collected solution is heated at 85° C. for 10 min to release the target-bound strands into solution, and then centrifuged at 13,000 rcf. The supernatant, containing library strands that bind hEGFR, is collected and reverse-transcribed to complementary DNA (cDNA) using a kit from Applied Biosciences, and then amplified via PCR. These amplicons are then used as templates to create a new 2′-F RNA pool for the next round of selection. This process is iterated for five rounds. Using a previously described flow cytometry protocol, pool affinity is monitored every round by transcribing the RNA sequences and hybridizing them with the reverse primer labeled with fluorescein at its 5′end. Various concentrations of the probe-hybridized pool (0 -500 nM) is mixed with 10⁵ target cells in binding buffer and incubated for 1 hour at 37° C. The cells are then washed to remove unbound library strands and analyzed via flow cytometry with a BD Melody instrument, with 10⁴ events acquired for each sample. The proportion of cells bound to the fluorescent library constructs is used to determine the pool's K_{D}. For the final enriched pool, cross-reactivity towards the various cell lines is determined.

Upon the completion of Anchor-SELEX, the binding properties of the isolated hEGFR-binding aptamers are identified and characterized. First, high-throughput sequencing is performed to identify highly enriched aptamer candidates from the final SELEX pool. The binding affinity of these aptamers to different cell lines expressing wild-type and mutant isoforms of hEGFR is then determined in order to evaluate the capability of the aptamers to tolerate target mutations. Then, the binding affinity, kinetics, and multiparatopic binding capability of the aptamers are verified, for example, using surface plasmon resonance (SPR).

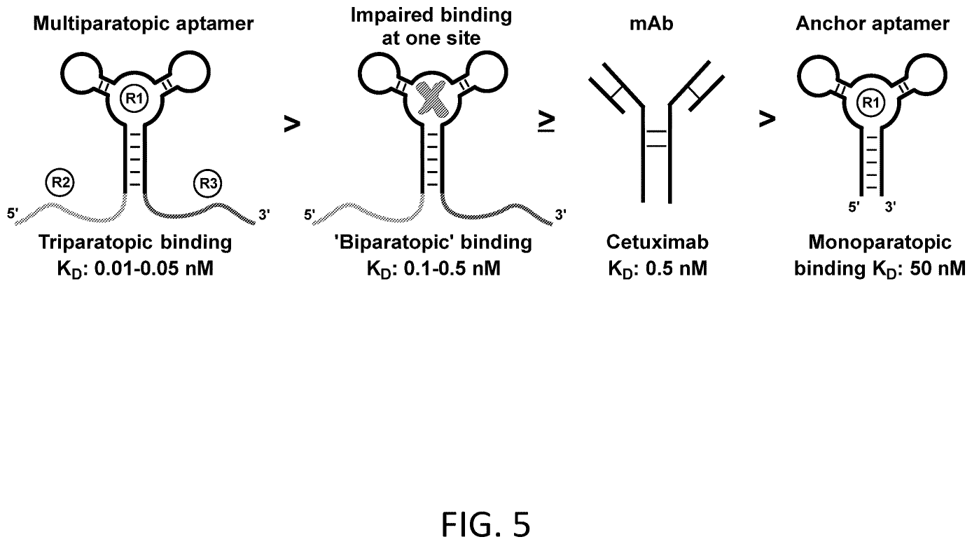

Additionally, the final enriched library pool is subjected to high-throughput sequencing. The five most abundant sequences are identified and synthesized without the PCR primer-binding region and with a 5′-FAM label (TriLink Biotechnologies). The binding affinity of these aptamer candidates is first tested for the five hEGFR variant-expressing cancer cell lines as well as the negative-control cell line (A549 EGFR knockout) used in Anchor-SELEX. Each cell line is grown to 90% confluence in plates, washed with binding buffer, and then incubated with 0 -100 nM 5′ FAM-labeled aptamer at 37° C. for 1 h. Cells are washed twice with DPBS to remove unbound aptamers. Flow cytometry is used to determine the proportion of FAM-labeled aptamer bound to cells, which is then used to determine K_{D}. As a comparison, the same procedure is repeated with 5′ FAM-labeled anchor aptamer and Cy 5.5-labeled cetuximab. Based on the 200-fold improvement in affinity observed with the biparatopic thrombin aptamer, it is predicted that the binding affinity of the triparatopic aptamer for these cell lines should be at least 50 pM, which is 1,000-fold improved relative to the anchor aptamer, and 10-fold improved compared to cetuximab (FIG. 5). To demonstrate the aptamer's robust tolerance for point mutations, flow cytometry is used to determine the aptamer's affinity to SW48 cells expressing hEGFR mutants that were not used during Anchor-SELEX, including I491M, S464L, and G465M, as well as HCC827 cells harboring an E746-A750 deletion. It is expected that aptamer binding may be reduced if these mutations are in or near the anchor aptamer-binding site, but the aptamer should retain affinity equivalent to cetuximab due to the preserved binding of the other two aptameric domains (FIG. 5).

The binding kinetics of the two triparatopic aptamers with the greatest affinity for all cell lines are further evaluated using the Biacore 3000 SPR instrument. hEGFR is modified onto a CM 5 dextran chip (Cytiva) via EDC-NHS chemistry. Unlabeled 2′-F aptamer (TriLink Biotechnologies) dissolved in binding buffer is flowed over the chip; after attaining equilibrium, the aptamer is washed away with binding buffer. The instrument records changes in the SPR resonance angle related to aptamer-target binding over time, and the resulting data can be used to determine binding stoichiometry, K_{D}, kₒₙ, and k_{off}. SPR is used to measure the binding properties of the original anchor aptamer and cetuximab. It is expected that the best triparatopic aptamer should exhibit a K_{D} and k_{off} for hEGFR that are improved by 1,000-fold relative to the anchor aptamer and 10-fold over cetuximab.

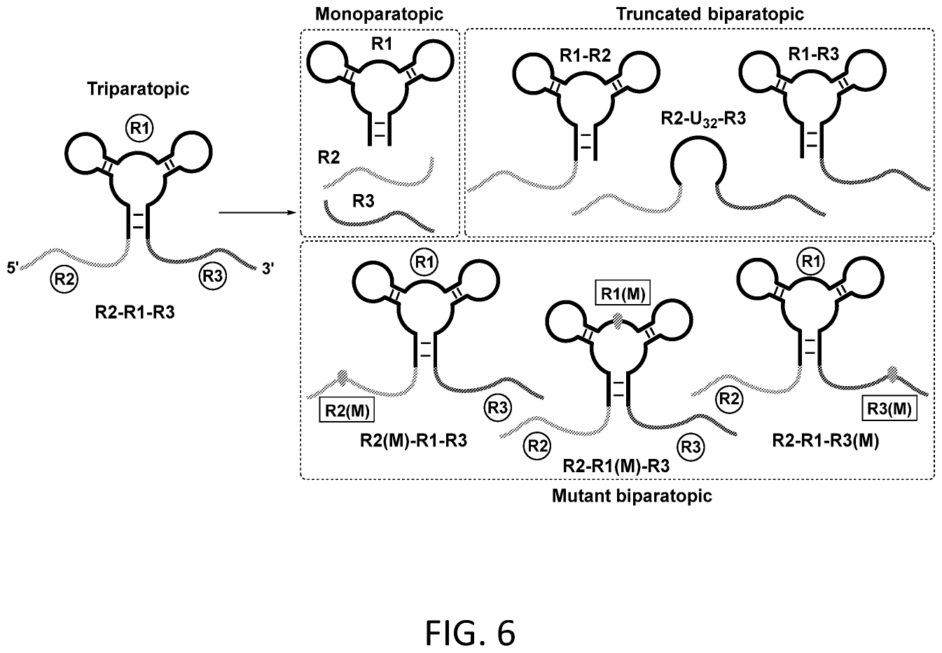

SPR will also be used to evaluate the binding contribution of each segment of the top-performing triparatopic aptamer. R1 is the core anchor aptamer, while R2 and R3 are the putative binding regions at the 5′ and 3′ ends, respectively. Nine constructs are derived from the triparatopic aptamer (FIG. 6): monoparatopic aptamers R1, R2, and R3, three biparatopic aptamers made by removing one of the binding domains from the triparatopic aptamer, and three biparatopic aptamers that have one point mutation at one of the three domains that impairs that particular domain's affinity. To confirm that all three regions can independently bind hEGFR, saturating concentrations of R1, R2, and R3 are sequentially flowed over the hEGFR-modified chip. If these aptamers can simultaneously bind hEGFR, the SPR resonance angle should consistently increase upon addition of each aptamer. Each construct's K_{D} and k_{off} is then measured using an hEGFR-modified chip. It is possible that truncated and mutated biparatopic aptamers have a poorer K_{D} and k_{off} than the native triparatopic aptamer, although they should still have a better K_{D} and k_{off} relative to the monoparatopic aptamers.

To evaluate the therapeutic efficacy of the aptamer and study its cell proliferation-inhibiting mechanism, a series of in vitro tests are performed. For example, experiments can first be conducted to confirm whether the aptamer can compete with hEGF to bind to cell-surface hEGFR. This is expected to occur given that the anchor aptamer has been previously demonstrated to prohibit binding of hEGF to hEGFR. About 10⁵ A549 cells expressing wild-type hEGFR are incubated with 1 nM 5′-FAM-labeled aptamer for 1 hour at 37° C. in binding buffer. Then, 0-100 nM TRITC-labeled hEGF is added to the cells and incubated for 1 hour at 37° C. to allow hEGF to displace the aptamer. The cells are then washed with DPBS to remove unbound aptamer and hEGF, and cells bound to either TRITC-hEGF or FAM-aptamer are counted using flow cytometry. As a control, the same protocol can be performed with a scrambled aptamer sequence labeled with 5′-FAM, which should have no affinity for hEGFR and thus not inhibit hEGF binding. The triparatopic aptamer can inhibit hEGF binding even at high concentrations due to its ultra-high affinity for hEGFR.

Additionally, experiments can be conducted to test the effect of the triparatopic aptamer on cell proliferation. The five hEGFR variant-expressing cell lines used as SELEX targets are cultured; media is replenished every other day, and the cells are passaged after reaching confluence. To evaluate the anti-proliferative effects of the aptamer, the cells are seeded in a 96-well plate at a density of 2,000 cells/well in their respective culture media and treated with 10 nM hEGF and 0-100 nM aptamer for 72 h at 37° C. Proliferation is measured using a 5-bromo-2′-deoxyuridine labeling and detection assay. The same test is performed with the scrambled triparatopic aptamer mutant as a negative control and cetuximab to compare against aptamer efficacy. The proliferation of cancer cells expressing wild-type or mutant hEGFR will be significantly reduced even at low concentrations of aptamer.

Next, the efficacy of the triparatopic aptamers is evaluated as a potential targeted therapeutic for adjuvant use with traditional cytotoxic chemotherapies. Experiments can be performed using a standard methyl thiazolyl tetrazolium (MTT) assay (Roche) on the five cancer cell lines used as SELEX targets using the chemotherapy agent 5-fluorouracil with or without the multiparatopic aptamer. Cells are transferred to 96-well plates at a seeding density of 5,000 cells/well in their respective culture medium for one day to allow adhesion. After rinsing with DPBS, the cells are incubated with a fixed concentration of aptamer (or DPBS for control) determined to have a significant effect on cell proliferation and 0-1,000 nM 5-fluorouracil for 72 h at 37° C. in cell culture medium. It is expected that the LC₅₀ (the 5-fluorouracil concentration resulting in 50% cell viability) for cancer cells treated in combination with the aptamer will be much lower than that of cancer cells treated with 5-fluorouracil alone, regardless of whether the cells express wild-type or mutant hEGFR.

In accordance with these embodiments, the present disclosure includes a method of identifying a multiparatopic aptamer comprising exposing a library of candidate multiparatopic aptamers to a target biomolecule, wherein each candidate multiparatopic aptamer in the library comprises an anchor aptamer capable of binding the target biomolecule and at least one candidate aptamer coupled to the anchor aptamer, and wherein the at least one candidate aptamer comprises a random nucleic acid sequence. The method also includes assessing binding of the candidate multiparatopic aptamer to the target biomolecule.

In some embodiments, the library of candidate multiparatopic aptamers is exposed to the target biomolecule in multiple rounds of selection. In some embodiments, the multiple rounds of selection comprise exposing the library of candidate multiparatopic aptamers to a variant of the target biomolecule in each round.

In some embodiments, the target biomolecule is a protein or polypeptide. In some embodiments, the protein or polypeptide is expressed by a cell (e.g., cell-SELEX method). In some embodiments, the protein or polypeptide is conjugated to a solid support. In other embodiments, the target biomolecule is a protein or polypeptide that is present in a solution and not bound to a solid support. In some embodiments, the target protein can be conjugated to or immobilized on a solid support such as a microbead (e.g., target-immobilized SELEX), including a streptavidin-linked microbead in which hybridization of a complementary DNA is labeled with biotin (e.g., library immobilized SELEX). In some embodiments, the target biomolecule is selected from the group consisting of cell surface receptors, endogenous biomarkers, cardiac proteins, oncoproteins, endocrine polypeptide hormones, gastrointestinal peptide hormones, cytokines, viral proteins, bacterial proteins, fungal proteins, and plant proteins. As would be recognized by one of skill in the art based on the present disclosure, the target molecule can include any peptide, polypeptide, or protein of interest, as the multiparatopic aptamers described herein can be selected to bind any epitope.

In some embodiments, assessing binding of the candidate multiparatopic aptamer to the target biomolecule comprises determining whether the K_{D} of the candidate multiparatopic aptamer is below a threshold. In some embodiments, the threshold K_{D} is at least 10-fold lower than the K_{D} of the anchor aptamer. In some embodiments, the threshold K_{D} is at least 11-fold lower than the K_{D} of the anchor aptamer. In some embodiments, the threshold K_{D} is at least 12-fold lower than the K_{D} of the anchor aptamer. In some embodiments, the threshold K_{D} is at least 13-fold lower than the K_{D} of the anchor aptamer. In some embodiments, the threshold K_{D} is at least 14-fold lower than the K_{D} of the anchor aptamer. In some embodiments, the threshold K_{D} is at least 15-fold lower than the K_{D} of the anchor aptamer. In some embodiments, the threshold K_{D} is at least 20-fold lower than the K_{D} of the anchor aptamer. In some embodiments, the threshold K_{D} is at least 50-fold lower than the K_{D} of the anchor aptamer. In some embodiments, the threshold K_{D} is at least 100-fold lower than the K_{D} of the anchor aptamer. In some embodiments, the threshold K_{D} is 100-fold or more lower than the K_{D} of the anchor aptamer.

In some embodiments, assessing binding of the candidate multiparatopic aptamer to the target biomolecule comprises performing surface plasmon resonance, isothermal titration calorimetry, microscale thermophoresis, biolayer interferometry, strand-displacement fluorescence assay, and intrinsic tryptophan fluorescence quenching, and any combination thereof. As would be recognized by one of ordinary skill in the art based on the present disclosure, other methods and assays can be used to assess the binding affinity/affinities of the candidate multiparatopic aptamer(s) to the target biomolecule.

What is claimed is:

1. A single-stranded nucleic acid molecule comprising:
an anchor aptamer capable of binding a target biomolecule; and
at least one candidate aptamer coupled to the anchor aptamer, wherein the at least one candidate aptamer comprises a random nucleic acid sequence.

2. The nucleic acid molecule of claim 1, wherein the anchor aptamer comprises a pre-determined nucleic acid sequence that binds an epitope on the target biomolecule.

3. The nucleic acid molecule of claim 1 or claim 2, wherein the anchor aptamer comprises a nucleic acid sequence that is from about 10 nucleotides to about 50 nucleotides in length.

4. The nucleic acid molecule of any one of claims 1 to 3, wherein the anchor aptamer comprises a nucleic acid sequence of a reference aptamer sequence.

5. The nucleic acid molecule of any one of claims 1 to 4, wherein the anchor aptamer comprises a nucleic acid sequence comprising at least one modification as compared to a reference aptamer sequence.

6. The nucleic acid molecule of claim 5, wherein the at least one modification comprises one or more of: (i) a substitution of one or more nucleotides; (ii) a deletion of one or more nucleotides; and/or (iii) an insertion of one or more nucleotides, as compared to a reference aptamer sequence.

7. The nucleic acid molecule of claim 6, wherein the anchor aptamer binds the target biomolecule with a K_{D} that is from about 10 nM to about 10 μM.

8. The nucleic acid molecule of any one of claims 1 to 7, wherein the nucleic acid molecule comprises two candidate aptamers flanking the anchor aptamer.

9. The nucleic acid molecule of any one of claims 1 to 8, wherein the at least one candidate aptamer comprises a random nucleic acid sequence that is determined to bind the target biomolecule.

10. The nucleic acid molecule of claim 9, wherein the at least one candidate aptamer binds an epitope on the target biomolecule that is different from the epitope to which the anchor aptamer binds.

11. The nucleic acid molecule of any one of claims 1 to 10, wherein the nucleic acid molecule binds the target biomolecule with a higher affinity than a reference anchor aptamer.

12. The nucleic acid molecule of any one of claims 1 to 11, wherein the nucleic acid molecule binds the target biomolecule with a K_{D} that is from about 1 fM to about 1 nM.

13. The nucleic acid molecule of any one of claims 1 to 12, wherein the at least one candidate aptamer comprises a nucleic acid sequence that is from about 10 nucleotides to about 50 nucleotides in length.

14. The nucleic acid molecule of any one of claims 1 to 13, wherein the nucleic acid molecule is from about 30 nucleotides to about 150 nucleotides in length.

15. The nucleic acid molecule of any one of claims 1 to 14, wherein the nucleic acid molecule is RNA, DNA, or combinations thereof.

16. The nucleic acid molecule of any one of claims 1 to 15, wherein the nucleic acid molecule comprises 2′-deoxy-2′-fluoro-ribonucleotides (2′-F RNA), a phosphorothioate modification, a 2′-O Methyl sugar, a LNA (locked nucleic acid), and a threose nucleic acid (TNA), and any combination thereof.

17. The nucleic acid molecule of any one of claims 1 to 16, wherein the nucleic acid molecule further comprises a linker.

18. The nucleic acid molecule of claim 17, wherein the nucleic acid molecule further comprises a linker, and wherein the linker facilitates binding of the nucleic acid molecule to a solid support.

19. The nucleic acid molecule of any one of claims 1 to 18, wherein the nucleic acid molecule further comprises a fluorescent tag or an electroactive tag.

20. The nucleic acid molecule of any one of claims 1 to 19, wherein the target biomolecule is a protein or polypeptide.

21. The nucleic acid molecule of any one of claims 1 to 20, wherein the target biomolecule is selected from the group consisting of cell surface receptors, endogenous biomarkers, cardiac proteins, oncoproteins, endocrine polypeptide hormones, gastrointestinal peptide hormones, cytokines, viral proteins, bacterial proteins, fungal proteins, and plant proteins.

22. A library comprising a plurality of any of the nucleic acid molecules of claims 1 to 21.

23. The library of claim 22, wherein the plurality of nucleic acid molecules comprises at least 10¹⁰ individual nucleic acid molecules, and wherein each sequence of the least one candidate aptamer is distinct.

24. A method of identifying a multiparatopic aptamer, the method comprising:
exposing a library of candidate multiparatopic aptamers to a target biomolecule, wherein each candidate multiparatopic aptamer in the library comprises an anchor aptamer capable of binding the target biomolecule and at least one candidate aptamer coupled to the anchor aptamer, wherein the at least one candidate aptamer comprises a random nucleic acid sequence; and
assessing binding of the candidate multiparatopic aptamer to the target biomolecule.

25. The method of claim 24, wherein the library of candidate multiparatopic aptamers is exposed to the target biomolecule in multiple rounds of selection.

26. The method of claim 25, wherein the multiple rounds of selection comprise exposing the library of candidate multiparatopic aptamers to a variant of the target biomolecule in each round.

27. The method of any one of claims 24 to 26, wherein the target biomolecule is a protein or polypeptide.

28. The method of claim 26, wherein the protein or polypeptide is expressed by a cell.

29. The method of claim 26, wherein the protein or polypeptide is conjugated to a solid support.

30. The method of claim 26, wherein the protein or polypeptide is present in a solution and not bound to a solid support.

31. The method of any one of claims 24 to 30, wherein assessing binding of the candidate multiparatopic aptamer to the target biomolecule comprises determining whether the K_{D} of the candidate multiparatopic aptamer is below a threshold.

32. The method of claim 31, wherein the threshold K_{D} is at least 10-fold lower than the K_{D} of the anchor aptamer.

33. The method of any one of claims 24 to 30, wherein assessing binding of the candidate multiparatopic aptamer to the target biomolecule comprises performing surface plasmon resonance, isothermal titration calorimetry, microscale thermophoresis, biolayer interferometry, strand-displacement fluorescence assay, and intrinsic tryptophan fluorescence quenching, and any combination thereof.
